(19) United States
(12) Patent Application Publication
(10) Pub. No.: US 2022/0409576 A1
(43) Pub. Date: Dec. 29, 2022

(54) NANO-PARTICULATE COMPOSITIONS FOR STIMULATING HOST INNATE IMMUNE RESPONSES FOR THERAPEUTIC APPLICATIONS

(71) Applicant: KENT STATE UNIVERSITY, Kent, OH (US)

(72) Inventors: Min-Ho KIM, Hudson, OH (US); Songping D. HUANG, Kent, OH (US); Bing YU, Stow, OH (US)

(21) Appl. No.: 17/765,262

(22) PCT Filed: Nov. 18, 2020
(86) PCT No.: PCT/US20/60958

§ 371 Date: Mar. 30, 2022

Related U.S. Application Data
(60) Provisional application No. 62/939,043, filed on Nov. 22, 2019.

(51) Int. Cl. A61K 31/366; A61K 9/16; A61K 31/365; A61P 31/04

(52) U.S. Cl. CPC A61K 31/366; A61K 9/1611; A61K 31/365; A61P 31/04

(57) ABSTRACT

Novel biocompatible Fenton-catalytic nano-particulate composites preferably based on nanoparticle (NP)-based catalysts, one or more reducing agents, and one or more peroxide compounds are formulated to take advantage of their ability to stimulate bactericidal as well as anti-tumor immune response by means of eliciting the generation of reactive oxygen species (ROS) in immune cells, in particular, in macrophages. The therapeutic composition can serve as a treatment for wound infections by, but not limited to, Staphylococcus aureus, Pseudomonas aeruginosa, Staphylococcus epidermidis, Klebsiella pneumoniae, and Acinetobacter baumannii, as a wound/lesion dressing that provide an anti-bacterial immune environment for the accelerated wound healing. In a similar principle, the therapeutic composition can serve as a treatment for solid tumors by providing an anti-tumor immune environment that inhibits tumor growth.

This invention was made with government support under Grant No. R01NR015674 awarded by National Institute of Health. The government has certain rights in the invention.

### FIELD OF THE INVENTION

Novel biocompatible Fenton-catalytic nano-particulate composites preferably based on nanoparticle (NP)-based catalysts, one or more reducing agents, and one or more peroxide compounds are formulated to take advantage of their ability to stimulate bactericidal as well as anti-tumor immune response by means of eliciting the generation of reactive oxygen species (ROS) in immune cells, in particular, in macrophages. The therapeutic composition can serve as a treatment for wound infections by, but not limited to, Staphylococcus aureus, Pseudomonas aeruginosa, Staphylococcus epidermidis. Klebsiella pneumoniae, and Acinetobacter baumannii, as a wound/lesion dressing that provide an anti-bacterial immune environment for the accelerated wound healing. In a similar principle, the therapeutic composition can serve as a treatment for solid tumors by providing an anti-tumor immune environment that inhibits tumor growth.

### BACKGROUND OF THE INVENTION

In response to a tissue infection, a large number of phagocytes such as neutrophils and macrophages infiltrate to the site of infection within a day of post-infection, where they phagocytose bacterial pathogens to resolve an infection. Staphylococcus aureus (S. aureus), P. aeruginosa, as well as Staphylococcus epidermidis, Klebsiella pneumoniae, and Acinetobacter baumannii are major human pathogens that cause persistent infections in skin and soft tissues. Upon phagocytosis of the pathogens, the production of sufficient quantities of ROS, in large part by mitochondrial respiratory chain, is critical for killing the pathogen by phagocytes. However, these bacteria have developed several strategies to escape ROS-mediated killing by eliciting a transcriptional regulation of ROS detoxifying enzymes. In that case, the bacteria can survive within the phagocytes by avoiding an attack by antibiotics, which often leads to the persistence of the bacteria that leads to chronic infection. Macrophages are key mediators of the immune response due to their characteristic to exhibit a phenotypic plasticity depending on environmental cues associated with various physiological and pathological conditions. In particular, a tightly regulated macrophage polarization toward an M1-like phenotype, characterized by the production of pro-inflammatory mediators and ROS, is critical for innate immune defense against bacterial pathogens. As such, insufficient ROS production in macrophages has been associated with a failure to kill the pathogen and persistency of infection. Especially when bacterial infections involve the formation of biofilm, macrophages are polarized towards an anti-inflammatory M2-like phenotype, which acts on attenuating the host immune responses. Therefore, a strategy to promote the generation of sufficient quantities of ROS in macrophages can be a potential therapeutic strategy to prevent dissemination of bacterial infections. It is logical that simple external interventions that stimulate host innate immune response to generate intracellular ROS in the macrophages will constitute novel approaches to treating bacterial infections, which can also be complementary to conventional antimicrobial approaches.

Macrophages comprise the most abundant population of immune cells in the tumor microenvironment (TME). In primary tumors and in metastatic sites, M2-like tumor-associated macrophages (TAMs) are implicated as mediators of tumor progression, invasion and metastasis. Although macrophages have the potential to kill tumor cells and to elicit anti-tumor reactions, signals or metabolic products derived from a hypoxic tumor environment were shown to drive the polarization of macrophages into M2-like TAMs, which contribute to tumor growth or recurrence by initiating anti-inflammatory and pro-tumor responses. In particular, presence of M2 macrophages and a high ratio of M2/M1 macrophages in the TME are clinically associated with poor prognosis in numerous types of solid tumors. The tumor-supporting functions of TAMs have been demonstrated for many types of malignancies, such as breast cancer, lung adenocarcinoma, cervical cancer, ovarian cancer, prostate cancer, melanoma, renal cell carcinoma, and esophageal cancer. In contrast, M1 polarized macrophages were shown to exhibit anti-tumor activity via iNOS-dependent generation of ROS and nitric oxide, which triggers the death of neighboring tumor cells by activation of the intrinsic apoptotic pathway.

### SUMMARY OF THE INVENTION

In this invention, we disclose the composition of Fenton-catalytic nano-particulate composites with properties of boosting host immune responses at the site of infection and solid tumors. The Fenton-catalytic nano-particulate composites comprise one or more NP-based catalysts, one or more reducing agents, and one or more peroxide compounds where they are mixed together at the optimal concentrations to boost immune cell (in particular macrophage)—mediated killing of bacterial cells and tumor cells. In this invention, we take advantage of highly phagocytic ability of macrophages that are capable of phagocytosing nanoparticles efficiently. Our nano-particulate composites proposed in the present invention are formulated to promote the killing of intracellular bacteria or tumor cells by means of stimulating a macrophage polarization towards the enhanced generation of ROS by triggering a Fenton-like reaction.

Among various forms of ROS, hydroxyl radical (OH⁻) is highly cytotoxic by causing oxidative damage to DNA and cell membrane. In the Fenton reaction-mediated generation of ROS, the oxidation of Fe²⁺ by hydrogen peroxide (H₂O₂) or an organic peroxide compound produces highly reactive hydroxyl radical. Hydrogen peroxide, a cellular metabolic byproduct, is susceptible to decomposition to produce the hydroxide anion OH⁻ and the hydroxyl free radical (OH⁻) in the presence of d-metal ions such as Fe²⁺ as the catalyst in the Fenton reaction:

Fe²⁺(aq)+H₂O₂(aq)=Fe³⁺(aq)+OH⁻(aq)+OH−(aq)

The above reaction raises the oxidation number of iron from +2 to +3. In the presence of a reducing agent, Fe³⁺ is reduced back to Fe²⁺ to form the active Fenton catalyst again, and thus the catalytic cycle can be sustained. Many intracellular antioxidants such as vitamins C and E, erythorbic acid, glutathione, as well as many natural antioxidants found in vegetables, fruits or green teas such as beta-carotene, flavonoids and polyphenols can all be effectively used as a reducing agent for such purpose. Besides Fe²⁺, almost all the transition metal ions (including Zn²⁺, Mg²⁺, Cr²⁺, Mn²⁺, Co²⁺, Ni²⁺, Cu⁺, etc.) with variable oxidation states can act as a catalyst for the above reaction. When these metal ions are used for the above reaction, this catalytic process is usually referred to as the Fenton-like reaction.

For biomedical applications of ROS generation, the free metal ions mentioned in the above and their small-molecule complexes are less desirable Fenton or Fenton-like catalysts. The main limitation is that the metal ions are readily subject to in vivo metal sequestration through chelation, and thus passivation of their catalytic activity, by various metal-binding biomolecules (heme molecules and carboxylic acids) and/or metal-binding proteins. Furthermore, such metal ions may also be adsorbed by tissues to enter the bloodstream, which will inadvertently increase the systemic levels of these metals. On the other hand, NPs incorporating such catalytically active transition metal ions are more suitable for the abovementioned applications, as they are more resistant to sequestration through chelation or adsorption by tissues. In this regard, iron oxide Fe₃O₄ NPs are seemingly the very promising choice for catalyzing the Fenton reaction because of their biocompatibility and low toxicity to the human body. Iron oxide Fe₃O₄ exists in nature as the mineral magnetite, and can be viewed as a composite oxide made up of iron(II) oxide FeO and iron(III) oxide Fe₂O₃. The better formula to reflect this fact is Fe²⁺ Fe³⁺₂O₄. As a mixed-valence compound, the intervalence charge transfer can readily occur between two iron sites with differing oxidation states in the crystal lattice of Fe²⁺ Fe³⁺₂O₄. This phenomenon hampers the redox reactivity of the Fe²⁺ site in the structure, and thus making this metal oxide much less catalytically active in the Fenton reaction. In fact, Fe²⁺ Fe³⁺₂O₄ belongs to a large class of minerals known as the spinel group with the general formula AB₂O₄ where A is typically a divalent metal ion such as Mg²⁺, Co²⁺, Cr²⁺, Mn²⁺, Fe²⁺, Cu²⁺, Ni²⁺ and Zn²⁺, B is usually a trivalent metal ion such as Al³⁺ and Fe³⁺ For example, the following iron spinels are all found in nature: magnesioferrite MgFe₂O₄, cuprospinel CuFe₂O₄, jacobsite MnFe₂O₄, trevorite NiFe₂O₄, zinc ferrite ZnₓFe₁₋ₓFe₂O₄ (0≤x≤1). The latter is in fact a solid solution where Zn²⁺ and Fe²⁺ are randomly distributed at the same crystallographic site, while crystal structure of the compound remains unchanged. Because of the disruption of intervalence charge transfer when a different divalent metal ion rather than Fe²⁺ occupies the octahedral holes in the spinel structure, the iron spinels containing Cu²⁺ or Mn²⁺ are catalytically more active than Fe₃O₄ for our intended applications. Besides, doping of Fe²⁺ ions into the crystal lattice of the above mentioned spinels to form solid solutions of FeₓA₁₋ₓFe₂O₄ (0≤x≤1), or more specifically FeₓMg₁₋ₓFe₂O₄ (0≤x≤1), FeₓCu₁₋ₓFe₂O₄(0≤x≤1), FeₓMn₁₋ₓFe₂O₄ (0≤x≤1), and FeₓZn_{1- x}Fe₂O₄ (0≤x≤1) can also effectively disrupt the intervalence charge transfer between Fe²⁺ and Fe³⁺, thus leading to the formation of better catalysts with even tunable catalytic activity. Preferably, “X” is (0.9≤x≤0.99).

Although hydrogen peroxide is produced in all cells including bacterial cells, its concentration is often too low to be therapeutically relevant because both enzymatic and non-enzymatic antioxidants present inside cells. Therefore, extracellular hydrogen peroxide must be administered to complete the catalytic cycles. Because hydrogen peroxide is not very stable when it is stored with a transition metal compound and a reducing agent, an organic peroxide compound is more preferable than hydrogen peroxide as an extracellular peroxide source.

An effective dose of a pharmaceutically accepted composition is administered in vivo to the site of infection via local administration to create an anti-bacterial immune environment for the inhibition of bacterial growth. Wherein, the site of infection indicates, but not limited to, infected wounds (diabetic ulcers, pressure ulcer, and venous ulcer) and biofilm-formed medical implants.

Similarly, an effective dose of a pharmaceutically accepted composition of the composite is administered in vivo to the site of solid tumors via local administration to create an anti-tumor immune environment for the inhibition of tumor growth. Wherein, the solid tumors indicate, but not limited to, breast cancer, lung adenocarcinoma, cervical cancer, ovarian cancer, prostate cancer, melanoma, or renal cell carcinoma, or any combination thereof.

### BRIEF DESCRIPTION OF THE DRAWINGS

FIG. 1 shows the effects of Fe₃O₄ iron oxide NPs (IONPs) on the generation of ROS and the on the bactericidal activity in RAW 264.7 macrophages against intracellular S. aureus. A. The quantification of ROS generation in the RAW 264.7 cells treated with varying concentration of IONPs (0-3 mg/mL). B. The colony number of viable bacteria (S. aureus) in the lysed RAW 264.7 macrophages treated with varying concentration of IONPs (0-3 mg/mL) following the exposure of live S. aureus.

FIG. 2 shows the synergistic effects of reducing agent, vitamin C (VC), and hydrogen peroxide (H₂O₂) on the IONPs-mediated generation of ROS and bactericidal activity in RAW 264.7 macrophages against intracellular S. aureus. (A-B) The quantification of ROS generation (A) and viable colony number of S. aureus (B) in the RAW 264.7 cells treated with VC (500 μM) alone, IONP (3 mg/mL) alone, or IONP (3 mg/mL)+VC (500 μM). (C-D) The quantification of ROS generation (C) and viable colony number of S. aureus (D) in the RAW 264.7 cells treated with IONP (3 mg/mL) alone, or IONP (3 mg/mL)+H₂O₂ (100 μM). E. Representative images of surviving S. aureus colony on agar plates from lysed RAW264.7 macrophages treated with H₂O₂ (100 μM), IONP (3 mg/mL), or IONP+H₂O₂. N=3-5 per group, *p<0.05

FIG. 3 shows the synergistic effects of IONP and vitamin C on triggering a Fenton reaction that generates hydroxyl radicals in RAW264.7 macrophages. A. The intracellular level of ferrous iron (Fe2+) in RAW 264.7 cells treated with IONPs (3 mg/mL) alone, IONPs with VC (500 μM), or IONPs with VC and BIP (iron chelator, 500 μM). B. The intracellular level of hydroxyl radical concentration in RAW 264.7 cells treated with IONPs (3 mg/mL) alone, IONPs with VC (500 μM), or in combination with BIP (500 μM) for 24 h. C. The bactericidal activity of RAW 264.7 cells treated with IONPs (3 mg/mL) with VC (500 μM), IONPs with VC and BIP (500 μM), or IONPs with VC and TIH (200 mM), assessed by antibiotic protection assay. *p<0.05, N=3-5 per group.

FIG. 4 shows the in vivo validation of the efficacy of IONPs, alone or in combination with reducing agent (VC) in the mouse model of wound S. aureus infection. A. The quantification of bacterial CFU number from wounds of C57BL/6 mice. *p<0.05, N=6 per group. S. aureus (1×10⁶ CFU/wound) was inoculated to 6-mm skin wounds at 0 day followed by topical application of IONPs or IONPsNC on each wound at 1 day. The skin wound tissues were dissected at 2 day for bacteria CFU counting and qPCR analysis. B. The expression of M1 marker (iNos and II-1β) and M2 marker (Arg-1 and Cd206) in the F4/80+ macrophages isolated from wounds mice treated with IONPs or IONPs with VC. *p<0.05, N=4 per group.

FIG. 5 shows a schematic on the proposed mechanism by which Fenton-catalytic nano-particulate composites can promote the killing of intracellular bacteria via triggering a Fenton reaction that generates intracellular ROS, in particular, hydroxyl radicals (OH⁻).

FIG. 6 shows a schematic of a liposome encapsulating the three ingredients of Fenton-catalytic nano-particulate composites.

### DETAILED DESCRIPTION OF THE INVENTION

The current invention comprises a composition containing a pharmaceutical or medical grade NP catalyst, reducing agent, and peroxide in an aqueous vehicle or hydrogel.

The one or more active NP catalyst contains nanoparticles E. G. FeₓA₁₋ₓFe₂O₄(O≤x≤1) where A is Cr²⁺, Co²⁺, or Ni²⁺, or one of the following solid solutions with the particles size in the size range of from about 2 nm to about 500 nm at concentrations between from about 0.1 mg/mL-100 mg/mL: (i) FeₓMg₁₋ₓFe₂O₄ (0≤x≤1), (ii) FeₓCu₁₋ₓFe₂O₄ (0≤x≤1), (iii) FeₓMn₁₋ₓFe₂O₄ (0≤x≤1), and (iv) FeₓZn₁₋ₓFe₂O₄ (0≤x≤1). Based on our cell culture experiment, the desirably concentration of NP catalyst is from about 1 to about 5 mg/mL and a size of from about 3 nm to about 120 nm., and preferably from about 4 nm to about 20 nm

The one or more reducing agents contain single or a combination of two or more the following reducing agents: (i) vitamin C, (ii) vitamin E, (iii) erythorbic acid (iv) glutathione, (v) citric acid, (vi) pyruvic acid, (vii) lactic acid, (viii) glucose, and (ix) erythrose, at concentrations in the range of 3 IM to 300 mM. Based on our cell culture experiment, the preferable concentration of reducing agent is from about 500 μM to about 1.5 mM. Among all these reducing agents, vitamin C and erythorbic acid exhibit the stronger reducing effect than the other. However: erythorbic acid is a synthetic stereoisomer of ascorbic acid and widely used as an antioxidant in processed foods. As such, the rate of metabolism for erythorbic acid in the human body is slower than that for vitamin C, which provides longer lasting reducing action. Hence, erythorbic acid is the preferred choice for this invention.

The one or more peroxide compound may be a single or a combination of two or more the following peroxo-containing agents: (i) hydrogen peroxide, (ii) benzoyl peroxide, (iii) acetyl benzoyl peroxide (acetozone), and (iv) artemisinin and derivatives thereof, and any combination thereof. The latter all contain an endoperoxide ring that makes the peroxo functional group much more stable than the normal open-chain peroxide compounds, and hence is the preferred choice for this invention. Furthermore, the concentration of peroxide compound is in the range of from about 3 μM to about 1,000 μM. Based on our cell culture experiment, the desired concentration of hydrogen peroxide is from about 100 μM to about 500 μM, and preferably from about 100 μM to about 300 mM.

The proof of principle for the application of Fenton-catalytic nanocomposite for treating bacterial infection was validated using in vitro culture models of macrophage-like RAW 264.7 cells and in vivo mouse model of skin wound infections by S. aureus, wherein Fe₃O₄ iron-oxide NP (IONP, 100 nm) and vitamin C (VC) were used as a NP catalyst and reducing agent, respectively. Then, we have examined if IONPs, alone or in combination with a VC or hydrogen peroxide, can be beneficial for macrophage-mediated bactericidal and pro-inflammatory immune responses against S. aureus.

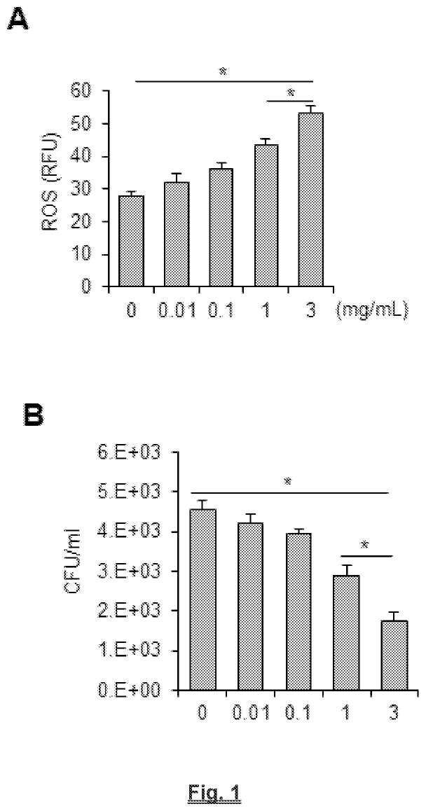

Once IONPs are internalized by macrophages, IONPs are degraded in endocytic organelles, resulting in the release of iron ion (Fe³⁺) in the cytoplasm. The newly formed iron ions can considerably affect the intracellular redox signaling that leads to the generation of ROS inside cells via a Fenton reaction. Thus, we investigated whether a IONPs-triggered Fenton reaction to generate ROS is sufficient to exhibit a bactericidal activity against Gram-positive bacteria, S. aureus, survived within macrophages. To ascertain this, we have assessed the capacity of IONPs to produce ROS in RAW 264.7 cells by treating the cells with varying concentrations of IONPs (0-3 mg/mL) and then quantifying the extent of total ROS generation using carboxy-H₂DCFDA, fluorogenic dye that can detect hydroxyl, peroxyl and other ROS activity within the cell. The levels of intracellular ROS in RAW 264.7 cells in response to IONPs were increased in a dose dependent manner (FIG. 1A). Importantly, the number of viable intracellular S. aureus was decreased with increasing concentrations of IONPs (FIG. 1B), which support that IONPs are capable of eliciting a bactericidal function of macrophages against intracellular S. aureus to some extent and this is associated with the capacity of IONPs to trigger the generation of ROS in macrophages.

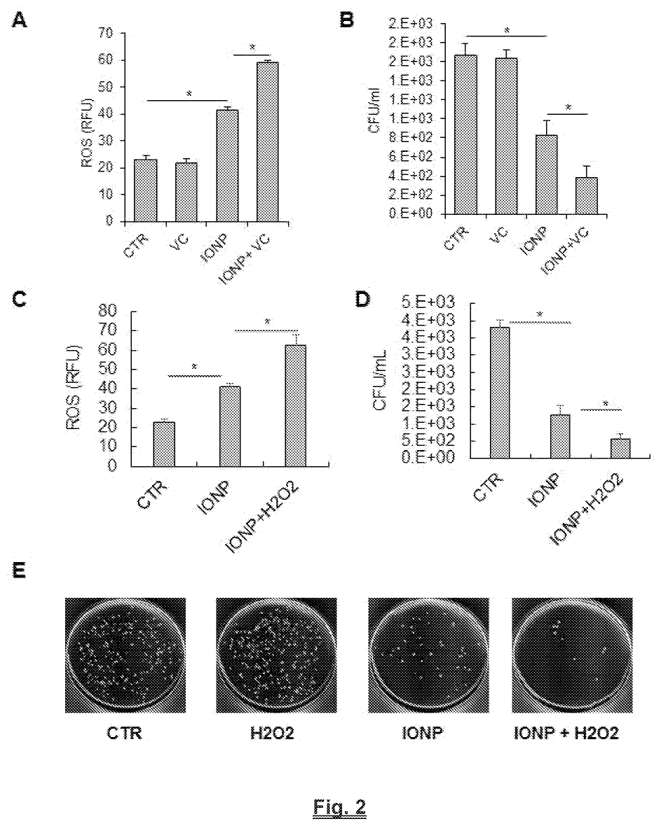

Since the ability to increase the availability of ferrous iron (Fe²⁺) in the cytoplasm is critical for ROS formation, the effect of reducing agents (VC) on the generation of ROS and bactericidal activity of macrophages was tested in macrophages. The combined treatment of IONPs with VC (500 μM) to RAW 264.7 macrophages could synergistically augment the generation of ROS (FIG. 2A), which was associated with increased bactericidal activity (FIG. 2B). Since the oxidation of Fe²⁺ by hydrogen peroxide (H₂O₂) produces highly reactive hydroxyl radical, the effect of hydrogen peroxide on the generation of ROS and bactericidal activity of macrophages was tested as well. Similar to the case of VC, the combined treatment of IONPs with VC (500 μM) to RAW 264.7 macrophages could significantly augment the generation of ROS (FIG. 2C), which was associated with increased bactericidal activity (FIGS. 2D and 2E).

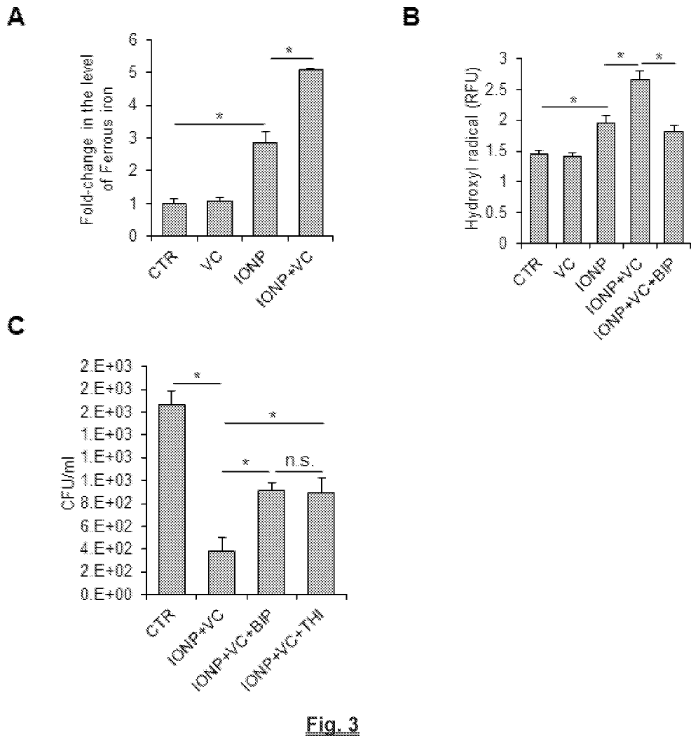

To further determine if the VC-mediated ROS generation and bactericidal activity were associated with a Fenton reaction due to increased release of Fe²⁺, the levels of Fe²⁺ were compared between RAW 264.7 cells treated with IONPs alone and IONPs with VC. The treatment of IONPs alone could significantly increase the level of Fe²⁺ in RAW 264.7 cells by 3-fold compared to the untreated cells, and its level was further augmented by 2-fold in the presence of VC, compared to IONPs only (FIG. 3A). Among various forms of ROS, hydroxyl radical (OH⁻) is highly cytotoxic by causing oxidative damage to DNA and cell membrane. In the Fenton reaction-mediated generation of ROS, the oxidation of Fe²⁺ by hydrogen peroxide (H₂O₂) produces highly reactive hydroxyl radical. To test if enhanced bactericidal activity of RAW264.7 cells with IONPs in combination with VC could be a consequence of Fe²⁺ release and the generation of hydroxyl radicals, the extent of hydroxyl radical generation and bactericidal activity in the RAW 264.7 cells treated with IONP and VC were quantified using the chelator of Fe²⁺, BIP, or scavenger of hydroxyl radicals, thiourea (THI) The treatment of BIP to RAW 264.7 cells significantly decreased IONPs and VC-induced generation of hydroxyl radicals up to the level of IONPs treatment only (FIG. 3B), which was associated with a decrease in bactericidal activity of RAW 264.7 cells (FIG. 3C). Additionally, the inhibition of hydroxyl radical formation by BIP was sufficient to reverse IONPs/VC-induced bactericidal activity of RAW 264.7 cells, which was comparable to the level induced by THI treatment. Taken together, these results support the synergistic effect of IONPs and VC in triggering a Fenton-like reaction in the macrophages, which contributed to the killing of intracellular bacteria.

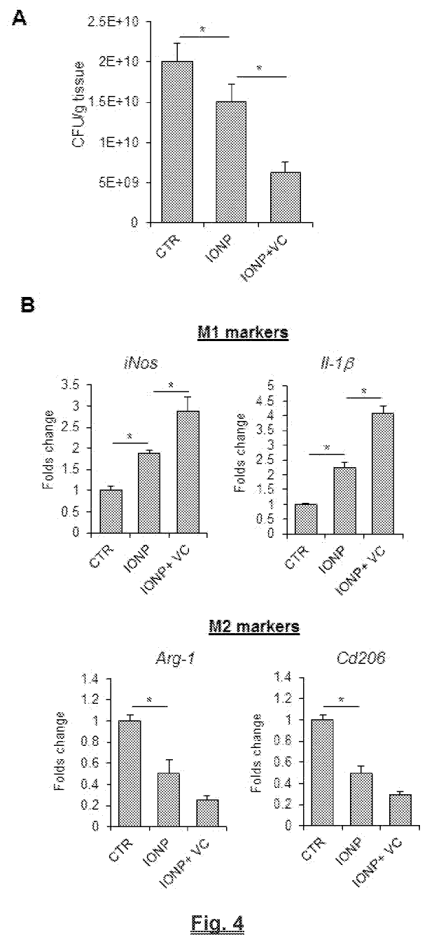

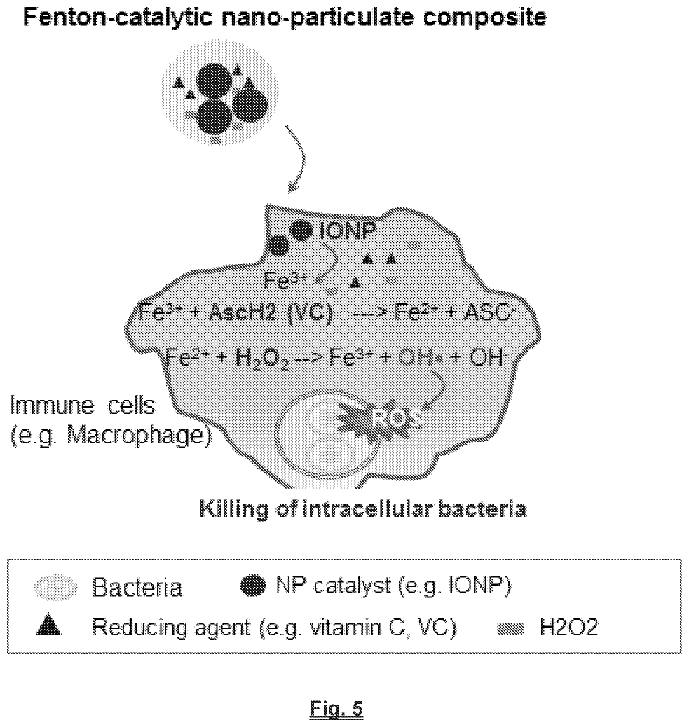

By observing the capacity of IONPs, in combination with VC, in promoting the bactericidal activity of RAW 264.7 cells in vitro, its efficacy was validated in vivo using a murine model of wound infection by S. aureus. The viable number of S. aureus was quantified from the wounded skin harvested at day 2 post-infection (FIG. 4A). The treatment of IONPs to the wound could reduce a bacterial burden by 25% compared to the control group (p<0.05). In consistence with our results from the in vitro study, the co-treatment of VC (500 μM) with IONPs significantly reduced S. aureus numbers in the wound by 75% compared to the control group. The F4/80⁺ macrophages from wounds of mice treated with IONPs and VC exhibited a significantly increased expression of iNos and II-1β compared to either IONPs alone or the untreated control group, which was associated with an attenuated expression of M2 markers including Arg-1 and Cd206 (FIG. 4B). Taken together, our results support that Fenton-catalytic nano-particulate composites can promote a bactericidal activity of macrophages by triggering a Fenton-like reaction (FIG. 5).

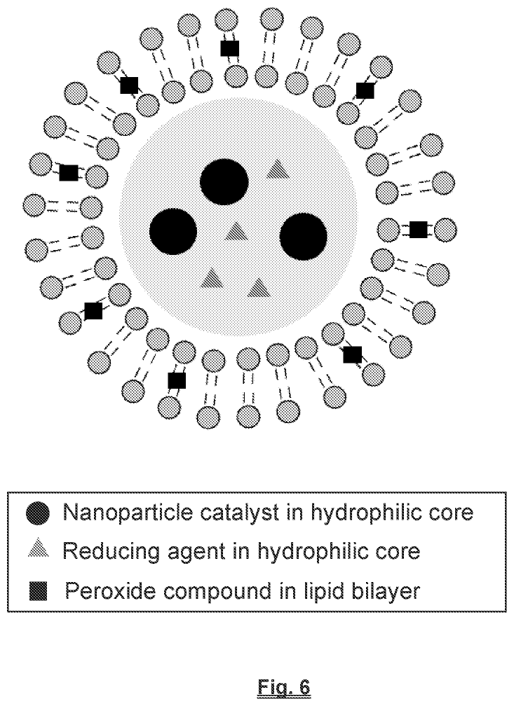

To improve the topical or intravenous delivery of the biocompatible Fenton-catalytic nano-particulate composites, a liposome encapsulating the three ingredients can be used as a drug-carrying vehicle to administrate the drug. A liposome is a spherical vesicle consisting of single or multiple lipid bilayers of phospholipids, for example, phosphatidylcholine or egg phosphatidylethanolamine. The aqueous solution core of a properly prepared liposome is surrounded by a hydrophobic lipid bilayer. Such structure allows the water-soluble nanoparticle-based Fenton catalyst and the reducing agent to be encapsulated in hydrophilic core, and on the other hand, the oil-soluble peroxide compound to be encapsulated in lipid bilayer (FIG. 6). The lipid bilayer to fuse with the biological cell membrane to enhance drug delivering efficiency. Such liposomes can be readily prepared by the thin-film hydration method followed by sequential extrusion. The following procedure is provided as an example for the encapsulation of the biocompatible Fenton-catalytic nano-particulate composites: a 50-mL chloroform solution containing 1,2-dipalmitoyl-sn-glycero-3-phosphocholine (DPPC) and cholesterol (Chol) in the mass ratio of 5:1 (the total lipid weight=10 mg) is added into a round-bottomed flask and heated at 50° C. in a water bath to remove the chloroform form the lipid film by a rotary evaporator. The film formed at the bottom of the flask is further evaporated under vacuum for 12 hours remove residual chloroform. The dry film was then hydrated by adding 1 mL of aqueous solution containing iron oxide nanoparticles (15 mg), vitamin C (30 mg) and artemisinin (15 mg) at 50° C. water bath for 30 min to produce liposomes loaded up with nano-particulate composites. The liposome dispersion is then homogenized with a mini extruder at 50° C. through a polycarbonate filter (average pore size=200 nm) for 20 times. Non-encapsulated iron oxide nanoparticles, vitamin C and artemisinin are removed by dialysis in a membrane dialysis bag.

While in accordance with the Patent Statutes, the best mode and preferred embodiments have been set forth, the scope of the invention is not limited thereto, but rather, by the scope of the attached claims.

What is claimed is:

1. A Fenton-catalytic nano-particulate composite comprising:
(a) one or more nanoparticle-based catalyst comprising FeₓA₁₋ₓFe₂O₄ (0≤x≤1) nanoparticles wherein A is Mg, Mn, Zn, Cu, Cr, Co, or Ni, or any combination of said different nanoparticles, and wherein, independently, said nanoparticles have a particle size of from about 2 nm to about 500 nm at a concentration of from about 0.1 to about 100 milli grams per milli liter;
(b) including one or more reducing agents, and
(c) including one or more peroxide compounds.

2. The composition of claim 1, wherein the amount of said one or more reducing agents is from about 3 μM to about 300 mM.

3. The composition of claim 2, wherein the amount of said one or more peroxides is from about 3 μM to about 1,000 μM.

4. The composition of claim 3, wherein said one or more reducing agents comprise (i) vitamin C, (ii) vitamin E, (iii) erythorbic acid (iv) glutathione, (v) citric acid, (vi) pyruvic acid, (vii) lactic acid, (viii) glucose, and (ix) erythrose, or any combination thereof.

5. The composition of claim 4, wherein said one or more peroxides comprise (i) hydrogen peroxide, (ii) benzoyl peroxide, (iii) acetyl benzoyl peroxide (acetozone), and (iv) artemisinin or any derivative thereof, or any combination thereof.

6. The composition of claim 5, wherein the amount of said one or more nanoparticles is from about 1 to about 5 milli grams per milli liter; wherein the size of said one or more nanoparticles is from about 3 nm to about 120 nm; and wherein the amount of said one or more reducing agent is from about 500 μM to about 1.5 mM.

7. The composition of claim 6, wherein the amount of said one more peroxides is from about 100 μM to about 500 μM.

8. The composition of claim 7, wherein said reducing agent comprises erythorbic acid.

9. The composition of claim 8, wherein said peroxide comprises artemisinin or a derivative thereof.

10. An aqueous solution comprising the composition of claim 1.

11. A hydrogel comprising the composition of claim 1.

12. A liposome comprising the composition of claim 1.

13. An aqueous solution comprising the composition of claim 9.

14. A hydrogel comprising the composition of claim 9.

15. A liposome comprising the composition of claim 9.

16. A method for treating infected wounds or lesions comprising the step of applying the composition of claim 1 to a wound or lesion.

17. A method of treating a solid tumor comprising the step of applying a local injection of the composition of claim 1 to said solid tumor.

18. The method of claim 16, wherein said infection comprises Staphylococcus aureus, Pseudomonas aeruginosa, Staphylococcus epidermidis, Klebsiella pneumoniae, and Acinetobacter baumannii, or any combination thereof.

19. The method of claim 17, wherein said tumor comprises breast cancer, lung adenocarcinoma, cervical cancer, ovarian cancer, prostate cancer, melanoma, or renal cell carcinoma, or any combination thereof.
